(19) United States
(12) Patent Application Publication
(10) Pub. No.: US 2022/0387688 A1
(43) Pub. Date: Dec. 8, 2022

(54) EQUIPMENT FOR THE CONTROL OF BIOMEDICAL DEVICES DURING EXTRACORPOREAL CIRCULATION AND RELATED SYSTEM

(71) Applicant: EUROSETS S.R.L., Medolla (IT)

(72) Inventors: Nicola GHELLI, Medolla (IT); Paolo FONTANILI, Medolla (IT); Marco CORBELLI, Medolla (IT)

(73) Assignee: EUROSETS S.R.L., Medolla (IT)

(21) Appl. No.: 17/775,989

(22) PCT Filed: Nov. 12, 2020
(86) PCT No.: PCT/IB2020/060654

§ 371 Date: May 11, 2022

(30) Foreign Application Priority Data
Nov. 12, 2019 (IT) 102019000021012

(51) Int. Cl. A61M 1/36

(52) U.S. Cl. CPC A61M 1/3639; A61M 2205/3331

(57) ABSTRACT

Equipment for the control of biomedical devices during extracorporeal circulation. The equipment includes at least one basic structure and a (e.g., tangible) controller or a control unit/device/component/etc. that is assembled on the basic structure and provided with at least one control unit/etc. operationally connectable to at least one biomedical device for extracorporeal circulation and configured to control it. The controller/etc. comprise at least one emergency control unit, operationally connectable to the biomedical device and configured to control the latter following a malfunction of the control unit.

### TECHNICAL FIELD

The present invention relates to a piece of equipment for the control of biomedical devices during extracorporeal circulation and a related system.

### BACKGROUND ART

As it is well known, for certain surgical operations, during which the functions of the patient's heart are temporarily suspended, extracorporeal blood circuits are created that involve the use of biomedical devices, such as e.g. the so-called “heart-lung” machines.

The heart-lung machines comprise a series of devices including a filtration device (also called “venous reserve”) adapted to filter the blood coming from the patient, a heat exchanger adapted to regulate the temperature of the blood coming out of the filtration device, and an oxygenator adapted to provide the correct supply of oxygen to the blood intended to be fed back into the patient. In particular, the blood arriving from the patient is conveyed to the oxygenator by means of a relevant pumping unit, which generally comprises a centrifugal pump of the dragging or magnetic levitation type, i.e. provided with a rotor element adapted to convey the incoming blood to the oxygenator as a result of the rotation thereof and with a stator element adapted to control the rotor element in rotation.

During extracorporeal circulation, a number of parameters must be monitored and controlled, including the number of revolutions of the rotor element and the flow rate of the blood conveyed to the oxygenator.

In this regard, the equipment of known type comprises control means configured to control the biomedical device and to monitor the operation thereof.

This piece of equipment is nowadays particularly used also in the extra-hospital field since the extracorporeal circulation is also applied in emergency situations, e.g. in roadside assistance, during which there is the need to adequately support the various devices in a dynamic and flexible manner

The equipment of known type for the control of biomedical devices and the relevant systems do have some drawbacks.

In particular, they are significantly affected by any malfunction of the control means.

In fact, as a result of a malfunction of the control means, the equipment of known type must be promptly replaced with a piece of equipment which is functioning.

A replacing piece of equipment is however not always available and such replacement takes a not negligible amount of time during which the patient's safety is put at risk.

This drawback makes known types of equipment particularly unreliable and risky.

### DESCRIPTION OF THE INVENTION

The main aim of the present invention is to devise a piece of equipment and a related system that allow ensuring the correct operation of the biomedical device, of the equipment itself and of the relevant system also as a result of a malfunction of the control means.

Another object of the present invention is to devise a piece of equipment for the control of biomedical devices during extracorporeal circulation and a related system that allow overcoming the above mentioned drawbacks of the prior art within a simple, rational, easy, effective to use and low cost solution.

The objects set out above are achieved by the present piece of equipment for the control of biomedical devices during extracorporeal circulation having the characteristics of claim 1.

The objects set out above are achieved by the present system for the control of biomedical devices during extracorporeal circulation having the characteristics of claim 8.

### BRIEF DESCRIPTION OF THE DRAWINGS

Other characteristics and advantages of the present invention will be more evident from the description of a preferred, but not exclusive, embodiment of a piece of equipment for the control of biomedical devices during the extracorporeal circulation and of a related system, illustrated by way of an indicative, yet non-limiting example, in the attached tables of drawings in which:

FIG. 1 is a schematic axonometric view of the system according to the invention;

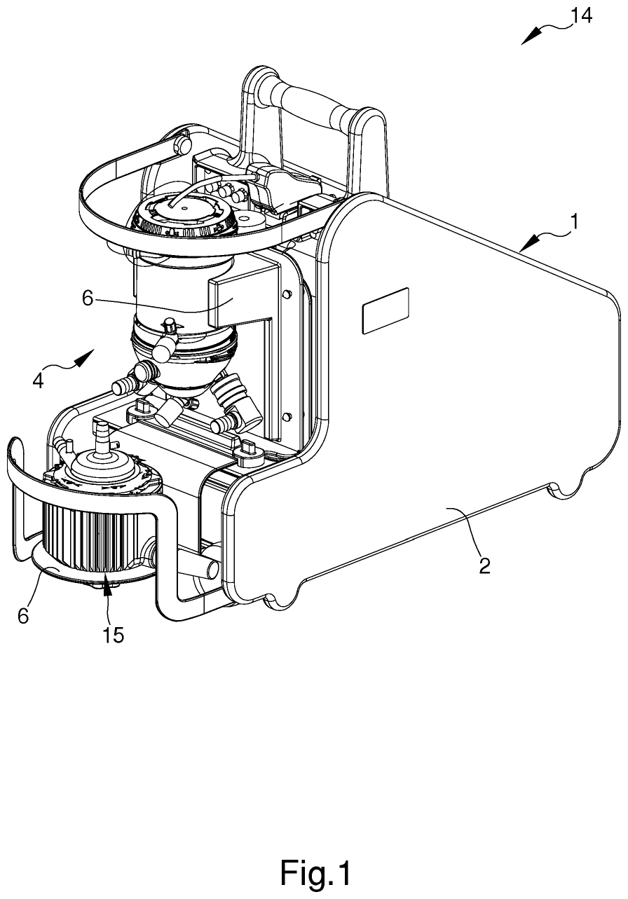

FIG. 2 is a block diagram of the system according to the invention in a first working configuration;

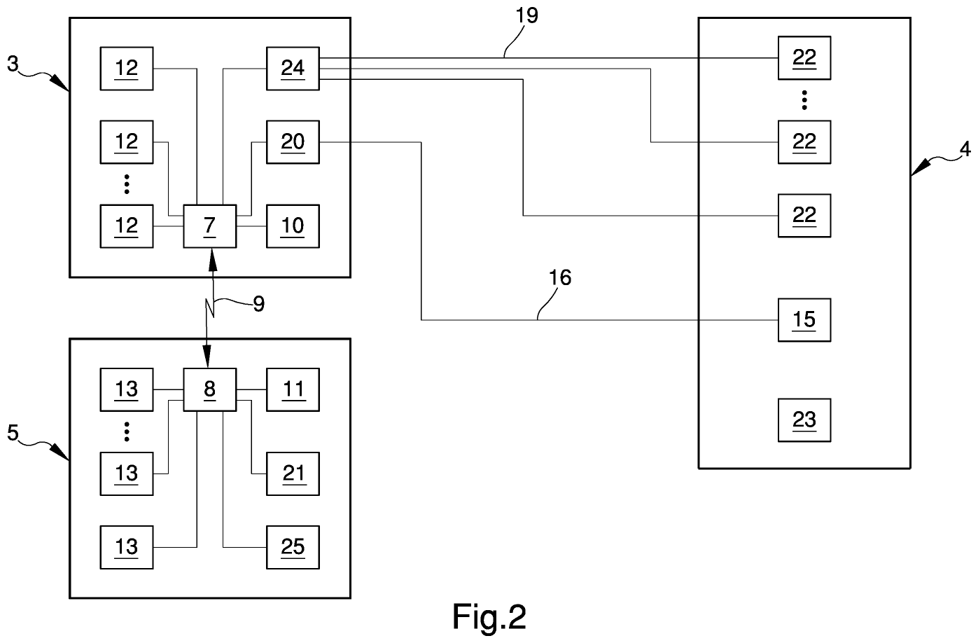

FIG. 3 is a block diagram of the system according to the invention in a second working configuration.

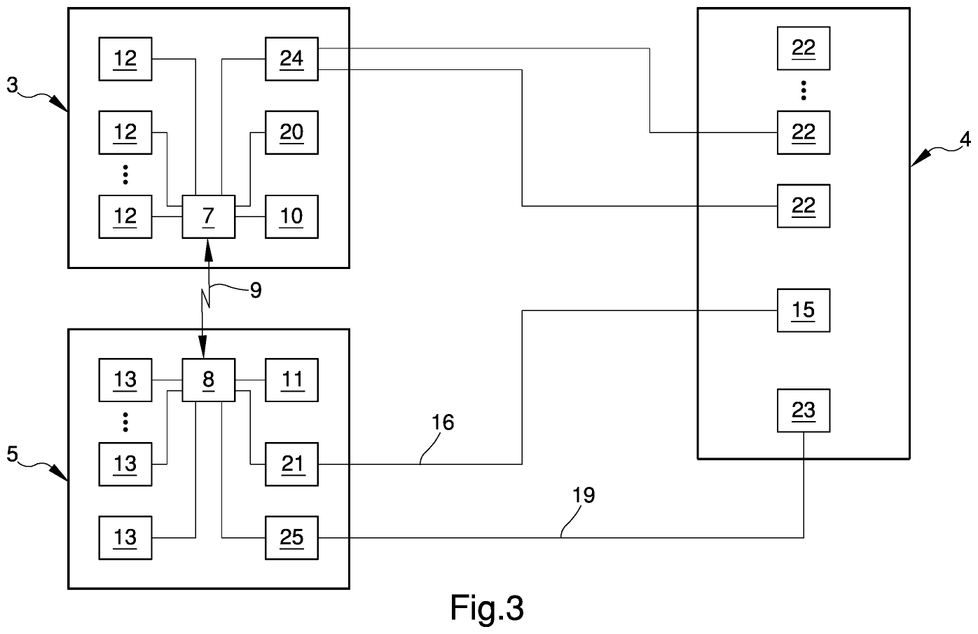

### EMBODIMENTS OF THE INVENTION

With particular reference to these figures, reference numeral 1 globally indicates a piece of equipment for the control of biomedical devices during the extracorporeal circulation.

The piece of equipment 1 for the control of biomedical devices during the extracorporeal circulation comprises:
at least one basic structure 2;
control means 3, 5, 9, 10, 11 of the piece of equipment 1 assembled on the basic structure 2 and provided with at least one control unit 3 operationally connectable to at least one biomedical device 4 for the extracorporeal circulation and configured to control the latter.

Advantageously, the control means 3, 5, 9, 10, 11 comprise at least one emergency control unit 5, operationally connectable to the biomedical device 4 and configured to control the latter as a result of a malfunction of the control unit 3.

This way, if the control unit 3 stops operating properly, the functionality of the biomedical device 4 is ensured by the emergency control unit 5.

Conveniently, the basic structure 2 comprises support means 6 of the biomedical device 4 adapted to fasten the latter to the same basic structure 2 in a removable manner.

Conveniently, the control means 3, 5, 9, 10, 11 comprise communication means 9 between the control unit 3 and the emergency control unit 5.

In addition, at least one of either the control unit 3 or the emergency control unit 5 is configured to check the malfunction of the other.

Preferably, according to the invention, the control unit 3 and the emergency control unit 5 are configured to check the malfunction of the other.

This way, any malfunction of the control unit 3 is immediately detected by the emergency control unit 5.

In addition, this device makes it possible to immediately identify any malfunction of the emergency control unit 5 during the correct operation of the control unit 3.

This way, it is possible to intervene promptly with the repair of the emergency control unit 5 and to prevent the emergency control unit 5 from malfunctioning and therefore being unusable as a result of the malfunction of the control unit 3, putting the patient's safety at risk.

In particular, the control unit 3 comprises at least one central unit 7 for the management of the same control unit and configured to check the malfunction of the emergency control unit 5.

Similarly, the emergency control unit 5 comprises at least one central emergency unit 8 for the control of the same emergency control unit and configured to check the malfunction of the control unit 3.

Therefore, according to the invention, the communication means 9 are operationally connected to the central unit 7 and to the central emergency unit 8 in order to allow these to check the malfunction of the emergency control unit 5 and of the control unit 3 respectively.

Preferably, the central unit 7 is an electronic device of the type e.g. of a microprocessor, microcontroller, PLC and the like.

Similarly, the central emergency unit 8 is an electronic device of the type e.g. of a microprocessor, microcontroller, PLC, control unit and the like.

In addition, the communication means 9 are preferably of the type of a low-voltage two-way electronic link for signal exchange.

Advantageously, the control means 3, 5, 9, 10, 11 comprise signaling means 10, 11 for signaling the malfunction of at least one of either the control unit 3 or the emergency control unit 5.

Preferably, the control unit 3 comprises at least one signaling unit 10 configured to notify a user of the malfunction of the emergency control unit 5.

Likewise, the emergency control unit 5 preferably comprises at least one emergency signaling unit 11 configured to notify a user of the malfunction of the control unit 3.

In addition, preferably, the signaling unit 10 is operationally connected to the central unit 7.

Likewise, the emergency signaling unit 11 is operationally connected to the central emergency unit 8.

Conveniently, the control unit 3 comprises at least one input/output device 12 configured to check/monitor the biomedical device 4.

In particular, the input/output device 12 is of the type selected from the list comprising a keyboard, a screen, a USB socket, a code reader, an electronic switch.

Preferably, the input/output device 12 is operationally connected to the central unit 7.

In addition, the control unit 3 comprises a plurality of input/output devices 12.

In particular, according to the invention, the control unit 3 comprises an input/output device 12 of each type included in the above mentioned list.

Conveniently, the emergency control unit 5 comprises at least one emergency input/output device 13 of the type selected from the list comprising a keyboard, a screen, a USB socket, a code reader, an electronic switch.

Preferably, the emergency input/output device 13 is operationally connected to the central emergency unit 8.

Preferably, the control unit 3 comprises a plurality of emergency input/output devices 13.

In particular, according to the invention, the emergency control unit 5 comprises an emergency input/output device 13 of each type included in the above mentioned list.

This way, the control unit 3 and the emergency control unit 5 have the same input/output devices for the correct management of the same equipment and of the biomedical device 4.

Conveniently, the equipment 1 comprises connection means 16, 19 of the biomedical device 4 to one of either the control unit 3 or the emergency control unit 5.

In particular, the connection means 16, 19 are connectable in a removable manner between at least a first operating configuration, wherein they operationally connect the biomedical device 4 to the control unit 3, and at least a second operating configuration, wherein they operationally connect the biomedical device 4 to the emergency control unit 5.

This way, as a result of a malfunction of the control unit 3, the biomedical device 4 is controlled by the emergency control unit 5, which ensures the correct operation of the biomedical device 4.

Advantageously, the connection means 16, 19 are operationally connectable to a plurality of components 15, 22, 23 of the biomedical device 4.

In particular, the connection means 16, 19 connect, in a removable manner, at least one component 15, 22, 23 to the control unit 3 and to the emergency control unit 5 in the first and in the second operating configuration, respectively. This way, the connection means 16, 19 allow each of the components 15, 22, 23 to be connected to the emergency control unit 5 in the event of a malfunction in the control unit 3.

This way, the equipment 1 allows ensuring the operation of the biomedical devices 4 provided with a plurality of components 15, 22, 23 which are essential to ensure proper extracorporeal circulation.

The present invention also relates to a system for extracorporeal circulation globally indicated with reference number 14.

The system 14 for extracorporeal circulation comprises:
at least one biomedical device 4 for the extracorporeal circulation;
at least one piece of equipment 1 having one or more of the characteristics of the equipment 1 described above.

Advantageously, the biomedical device 4 comprises at least one pumping motor-driven unit 15 for extracorporeal circulation.

In addition, the connection means 16, 19 comprise at least one connecting line 16 which operationally connects the motor-driven unit 15 to the control unit 3 and to the emergency control unit 5 in the first and in the second operating configuration, respectively.

Preferably, the connecting line 16 can be manually connected/disconnected by a user who, as a result of a malfunction of the control unit 3, disconnects the connecting line 16 from the latter and connects it to the emergency control unit 5.

Furthermore, the connecting line 16 is of the type of an electronic connection device provided with connectors such as, e.g, an electric cable or the like.

Advantageously, the control unit 3 comprises at least one drive unit 20 of the motor-driven unit 15 and the emergency control unit 5 comprises at least one emergency drive unit 21 of the motor-driven unit 15.

This way, the connecting line 16 operationally connects the motor-driven unit 15 to the drive unit 20 and to the emergency drive unit 21 in the first operating configuration and in the second operating configuration, respectively.

In particular, the drive unit 20 is operationally connected to the central unit 7. Similarly, the emergency drive unit 21 is operationally connected to the central emergency unit 8.

Conveniently, the biomedical device 4 comprises at least one monitoring unit 22, 23 of the extracorporeal circulation provided with at least one sensor device 22 selected from the list comprising a flow meter, pressure sensor, level sensor, in-line air detector, venous probe, arterial probe.

In addition, the connection means 16, 19 comprise at least one linking line 19 which operationally connects the sensor device 22 to the control unit 3 and to the emergency control unit 5 in the first operating configuration and in the second operating configuration, respectively.

Conveniently, the linking line 19 can be manually connected/disconnected by a user, who, as a result of a malfunction of the control unit 3, disconnects the linking line 19 from the latter and connects it to the emergency control unit 5. This way, as a result of a malfunction of the control unit 3, the sensor device 22 is controlled by the emergency control unit 5, which uses the data collected by this sensor to ensure the correct extracorporeal circulation.

Preferably, the sensor device 22 is of the type of a flow meter.

Conveniently, the monitoring unit 22, 23 comprises a plurality of sensor devices 22.

Preferably, the monitoring unit 22, 23 comprises at least one sensor device 22 for each type included in the list comprising a pressure sensor, level sensor, in-line air detector, venous probe, arterial probe.

In addition, preferably, the monitoring unit 22, 23 comprises three sensor devices 22 of the type of pressure sensors.

Advantageously, at least one of the sensor devices 22 is operationally connected to the control unit 3.

In particular, according to the invention, all the sensor devices 22 with the exception of the flow meter, are operationally connected in a fixed manner to the control unit 3.

In other words, according to the invention, only the sensor device 22 of the type of a flow meter can be connected in a removable manner to one of either the control unit 3 or the emergency control unit 5 by interposition of the linking line 19. On the contrary, the other sensor devices 22 are operationally permanently connected to the control unit 3, i.e. there is no provision for these sensors to be disconnected from the latter.

Further embodiments of the system 14 cannot however be ruled out wherein the connection means 16, 19 comprise a plurality of linking lines 19, each adapted to connect a respective sensor device 22 to the control unit 3 and to the emergency control unit 5 in the first operating configuration and in the second operating configuration, respectively.

Conveniently, the monitoring unit 22, 23 comprises at least one emergency sensor device 23 selected from the list comprising a flow meter, pressure sensor, level sensor, in-line air detector, venous probe, arterial probe.

In addition, the linking line 19 operationally connects the control unit 3 to the sensor device 22 in the first operating configuration, and operationally connects the emergency control unit 5 to one of either the sensor device 22 or the emergency sensor device 23 in the second operating configuration.

Preferably, the emergency sensor device 23 is a flow meter.

According to the embodiment of the system 14 shown in the figures, in the first operating configuration the linking line 19 operationally connects the control unit 3 to the sensor device 22 and, in the second operating configuration, the linking line 19 operationally connects the emergency sensor device 23 to the emergency control unit 5.

Further embodiments of the system 14 cannot however be ruled out wherein, in the first operating configuration, the linking line 19 operationally connects the control unit 3 to the sensor device 22 and, in the second operating configuration, the linking line 19 connects the same sensor device to the emergency control unit 5.

In other words, as a result of a malfunction of the control unit 3, the linking line 19 must be disconnected from the control unit 3 in order to be connected to the emergency control unit 5 and connect the latter to the sensor device 22 previously connected to the control unit 3.

In addition, further embodiments of the system 14 cannot be ruled out in which the monitoring unit 22, 23 comprises a plurality of emergency sensor devices 23. For example, embodiments of the system 14 cannot be ruled out wherein the monitoring unit 22, 23 comprises one emergency sensor device 23 for each type listed above, or wherein the monitoring unit 22, 23 comprises emergency sensor devices 23 in the same number and of the same type as the sensor devices 22. Advantageously, the control unit 3 comprises at least one check unit 24 of the monitoring unit 22, 23.

In addition, the emergency control unit 5 comprises at least one emergency check unit 25 of the monitoring unit 22, 23.

This way, the linking line 19 operationally connects the monitoring unit 22, 23 to the drive unit 20 and to the emergency drive unit 21 in the first operating configuration and in the second operating configuration, respectively.

In particular, according to the invention, the sensor devices 22 other than the flow meter are operationally permanently connected to the check unit 24.

It has, in practice, been ascertained that the described invention achieves the intended objects.

In particular, the emergency control unit ensures the correct operation of the equipment and of the related system even in the event of a malfunction of the control unit.

In addition, the communication means make it possible to identify in advance any malfunction of the emergency control unit, thus reducing the risk of simultaneous malfunction of the control unit and of the emergency control unit.

1) Equipment for the control of biomedical devices during extracorporcal circulation, said equipment comprising:
at least one basic structure; and
control means assembled on said basic structure and provided with at least one control unit operationally connectable to at least one biomedical device for extracorporcal circulation and configured to and/or programmed to control said at least one biomedical devices, wherein
said control means comprise at least one emergency control unit, operationally connectable to said biomedical device and configured to and/or programmed control the biomedical device following a malfunction of said control unit.

2) The equipment according to claim 1, wherein said basic structure comprises support means of said biomedical device adapted to fasten the biomedical device to the same basic structure in a removable manner.

3) The equipment according to claim 1, wherein said control means comprise communication means between said control unit and said emergency control unit, at least one between the control unit and the emergency control unit being configured to check the malfunction of the other.

4) The equipment according to claim 1, wherein said control means comprise at least one input/output device configured to and/or programmed to check/monitor said biomedical device, said input/output device being of a type selected from a list comprising at least a keyboard, a screen, a USB socket, a code reader, and an electronic switch.

5) The equipment according to claim 1, wherein said emergency control unit comprises at least one emergency input/output device configured to and/or programmed to check/monitor said biomedical device, said emergency input/output device being of a type selected from a list comprising at least a keyboard, a screen, a USB socket, a code reader, and an electronic switch.

6) The equipment according to claim 1, further comprising connection means of said biomedical device to one of either said control unit and said emergency control unit, wherein said connection means being connectable in a removable manner between at least a first operating configuration, wherein said connection means operationally connect said biomedical device to said control unit, and at least a second operating configuration, and wherein said connection means operationally connect said biomedical device to said emergency control unit.

7) The equipment according to claim 6, wherein said connection means are operationally connectable to a plurality of components of said biomedical device, said connection means connecting in a removable manner at least one component to said control unit and to said emergency control unit in said first and in said second operating configuration, respectively.

8) System for the control of biomedical devices during extracorporeal circulation, said system comprising:
at least one biomedical device for extracorporeal circulation; and
at least one piece of said equipment according to claim 1.

9) The system according to claim 8, wherein
said biomedical device comprises at least one pumping motor-driven unit for extracorporeal circulation; and
said connection means comprise at least one connecting line which operationally connects said motor-driven unit to said control unit and to said emergency control unit in said first and in said second operating configuration respectively.

10) The system according to claim 8, wherein
said biomedical device comprises at least one monitoring unit of the extracorporeal circulation provided with at least one sensor device selected from a list comprising at least a flow meter, a pressure sensor, a level sensor, an in-line air detector, a venous probe, and an arterial probe; and
said connection means comprise at least one linking line which operationally connects the sensor device to said control unit and to said emergency control unit in said first and in said second operating configuration, respectively.

11) The system according to claim 10, wherein said monitoring unit comprises a plurality of said sensor devices.

12) The system according to claim 10, wherein
said monitoring unit comprises at least one emergency sensor device selected from a list comprising at least a flow meter, a pressure sensor, a level sensor, an in-line air detector, a venous probe, and an arterial probe; and
said linking line operationally connects said control unit to said sensor device in said first operating configuration, and operationally connects said emergency control unit to one of either said sensor device or said emergency sensor device in said second operating configuration.

13) Equipment for the control of biomedical devices during extracorporeal circulation, said equipment comprising:
at least one basic structure; and
a tangible controller assembled on said basic structure and provided with at least one control unit operationally connectable to at least one biomedical device for extracorporeal circulation and configured to and/or programmed to control said at least one biomedical device, wherein
said controller comprise at least one emergency control unit, operationally connectable to said biomedical device and configured to and/or programmed control the biomedical device following a malfunction of said control unit.
